(19) United States
(12) Patent Application Publication
(10) Pub. No.: US 2026/0360477 A1
(43) Pub. Date: Nov. 12, 2026

(54) SYSTEMS AND METHODS FOR PREDICTING ELECTRONIC STRUCTURES OF CHEMICAL SYSTEMS VIA GUASSIAN MULTIPOLE FEATURIZATION

(71) Applicant: Toyota Research Institute, Inc., Los Altos, CA (US)

(72) Inventors: Xiangyun Lei, Atlanta, GA (US); Shashikant Kumar, Atlanta, GA (US)

(73) Assignees: Toyota Research Institute, Inc., Los Altos, CA (US); Toyota Jidosha Kabushiki Kaisha, Toyota-shi Aichi-ken (JP)

(21) Appl. No.: 19/200,890

(22) Filed: May 7, 2025

(51) Int. Cl. G16C 20/30; G16C 20/70

(52) U.S. Cl. CPC G16C 20/30; G16C 20/70

(57) ABSTRACT

A system for predicting electronic structures for chemical systems includes a processor and a memory communicably coupled to the processor. The memory stores machine-readable instructions that, when executed by the processor, cause the processor to train a machine learning (ML) regression module with chemical systems and corresponding electronic structures of the chemical systems, generate a grid-level representation of a selected chemical system, generate Gaussian-multipole featurization vectors for the grid-level representation, and predict, using the trained ML regression model, an electronic structure for the selected chemical system.

### TECHNICAL FIELD

The present disclosure relates generally to electronic structures of chemical systems and particularly to predicting electronic structures and properties of chemical systems.

### BACKGROUND

The electronic structure of a chemical system, including the charge density and loss of density of states (LDOS), can be important in determining properties of the chemical system such as the dielectric constant and bandgap energy, among others. In addition, there are hundreds if not thousands of known chemical systems (e.g., materials such as oxides) that scientists could potentially synthesize, test and develop for electronic applications. However, determining the electronic structure of a chemical system using experimental techniques such as photoelectron spectroscopy (PES) and x-ray absorption spectroscopy (XAS) and/or using theoretical models with associated computation methods is time and cost intensive.

The present disclosure addresses issues related to determining electronic structures of chemical systems, and other issues related to electronic structures of chemical systems.

### SUMMARY

This section provides a general summary of the disclosure and is not a comprehensive disclosure of its full scope or all of its features.

In one form of the present disclosure, a system for predicting electronic structures for chemical systems includes a processor and a memory communicably coupled to the processor. The memory stores machine-readable instructions that, when executed by the processor, cause the processor to train a machine learning (ML) regression module with ground truth data that includes chemical systems and their known electronic structures. The memory also stores machine-readable instructions that, when executed by the processor, cause the processor to generate a grid-level representation of a selected chemical system for which the electronic structure is not known, generate featurization vectors for the grid-level representation, and predict, using the trained ML regression model, an electronic structure for the selected chemical system.

In another form of the present disclosure, a system for predicting electronic structures for chemical systems includes a processor and a memory communicably coupled to the processor. The memory stores machine-readable instructions that, when executed by the processor, cause the processor to train a ML regression module with grid-level Gaussian-multipole (GMP) featurization vectors for chemical systems and their known grid-level density functional theory (DFT) electronic structure data, generate a 3D grid-level representation of a selected chemical system for which the electronic structure is not known, generate GMP featurization vectors for the grid-level representation, and predict, using the trained ML regression model, grid-level DFT electronic structure data for the selected chemical system.

In still another form of the present disclosure, a system for predicting electronic structures for chemical systems includes a processor and a memory communicably coupled to the processor. The memory stores machine-readable instructions that, when executed by the processor, cause the processor to generate a 3D grid-level representation of a selected chemical system for which the electronic structure is not known, generate GMP featurization vectors for the grid-level representation, and predict, using a ML regression model trained with grid-level GMP featurization vectors and corresponding grid-level DFT electronic structure data for known chemical systems, grid-level electronic structure data for the selected chemical system. The corresponding DFT electronic structure data for the chemical systems includes at least one of DFT charge density data and DFT LDOS data, and the predicted grid-level electronic structure data for the selected chemical system includes at least one of charge density data and LDOS data.

Further areas of applicability and various methods of enhancing the above technology will become apparent from the description provided herein. The description and specific examples in this summary are intended for purposes of illustration only and are not intended to limit the scope of the present disclosure.

### BRIEF DESCRIPTION OF THE DRAWINGS

The present teachings will become more fully understood from the detailed description and the accompanying drawings, wherein:

FIG. 1 is a block diagram of a system for determining the electronic structure of a chemical system according to the teachings of the present disclosure;

FIG. 2A illustrates an atomic model of gallium (III) oxide (Ga₂O₃);

FIG. 2B illustrates a 3D grid-level representation overlayed onto the atomic model in FIG. 2 according to the teachings of the present disclosure;

FIG. 2C illustrates a 3D grid-level representation of Ga₂O₃ according to the teachings of the present disclosure;

FIG. 3 is a flow chart for training a machine learning regression model according to the teachings of the present disclosure;

FIG. 4 is a flow chart for a method of determining the electronic structure of a chemical system, and optionally a property of the chemical system, according to the teachings of the present disclosure.

### DETAILED DESCRIPTION

The present disclosure provides systems and methods for determining the electronic structure of a chemical system (also referred to herein as “material”). As used herein, the term “chemical system” and “material” refers to crystalline materials (e.g., oxides, nitrides, sulfides, intermetallics, among others), amorphous materials, liquids, and gases. The systems and methods include training a machine learning (ML) regression model with chemical systems and their corresponding known DFT electronic structure data. In some variations, systems and methods include training a machine learning (ML) model using a grid-level representations of ground truth chemical systems and featurization vectors generated for each grid point of the grid-level representations. The featurization vectors describe the local environment around a corresponding grid point and can be generated via a featurization scheme (i.e., a mathematical formula/algorithm).

Before and/or during training of the ML model, the ground truth chemical systems are simulated using density functional theory (DFT) computations such that electronic structure information (e.g., charge density and LDOS) is provided for the same grid-level representations and grid points noted above. In this manner, the ML model is trained with feature vectors as inputs and corresponding electronic structure information as targets. For example, in some variations, a regression model is trained to predict the known electronic structure information targets given the known featurization vectors as input.

After training, the ML model predicts electronic structure information for a selected chemical system that is not in or part of the ground truth chemical systems. Particularly, a grid-level representation is generated for the selected chemical system, as are featurization vectors for each grid point of the grid-level representation, and the featurization vectors are used as input for the trained ML model, which in turns predicts electronic information for the same grid points of the selected chemical system. And in some variations, the predicted electronic structure information for the grid points of the selected chemical system grid-level representation is used to calculate additional information/properties thereof using known quantum mechanical theory. Stated differently, the output of the trained ML model is used as input for one or more quantum mechanical theory algorithms/models. Non-limiting examples of such quantum mechanical theory algorithms/models include Kohn-Sham density functional theory algorithms/models for calculating chemical system energy, forces, bandgap, and density of states (DOS), and solid-state mechanics algorithms/models for calculating optimized atomic structure, phonon spectra, and dynamics.

As used herein, the phrase “selected chemical system” refers to a material that has been synthesized and/or predicted to exist via one or more computation methods, but for which the electronic structure has not been empirically determined and/or modeled via first principles modeling such as DFT, and for which the electronic structure is predicted using the systems and methods according to the teachings of the present disclosure. The phrase “electronic structure” as used herein refers to the arrangement and characteristics of electrons within a chemical system. Not being bound by theory, the electron configuration of an atom determines its chemical behavior, e.g., the atom's tendency to form bonds with other atoms. Also, when atoms combine to form a chemical system (e.g., an oxide), the orbitals of the atoms interact to form molecular or compound orbitals, and distribution of electrons in the compound orbitals determine the chemical system's overall electronic structure and properties such as electronic, magnetic, and optical properties. And as used herein, the phrase “electronic structure properties” refers to the measurable and observable consequences of a given electronic structure for a chemical system.

Accordingly, the systems and methods according to the teachings of the present disclosure do not provide or predict materials for which the atomic structure is not known, but provide or predict electronic structures and/or one or more electronic structure properties of materials for which the atomic structure is known in a computationally efficient manner compared to traditional DFT modeling/calculations. Stated differently, the systems and methods disclosed herein solve the problem of needing or requiring cost and time intensive empirical and/or computational methods to predict the electronic structures of known materials and thereby enhance the selection of materials for testing and possible commercial. For example, in some variations DFT modeling of the electronic structure of a selected chemical system (e.g., an oxide) can take hundreds, thousands, or even up to a million central processing unit (CPU) hours, while the systems and methods disclosed herein predict the electronic structure and/or one or more electronic structure properties of the same selected chemical system in orders of magnitude less CPU hours.

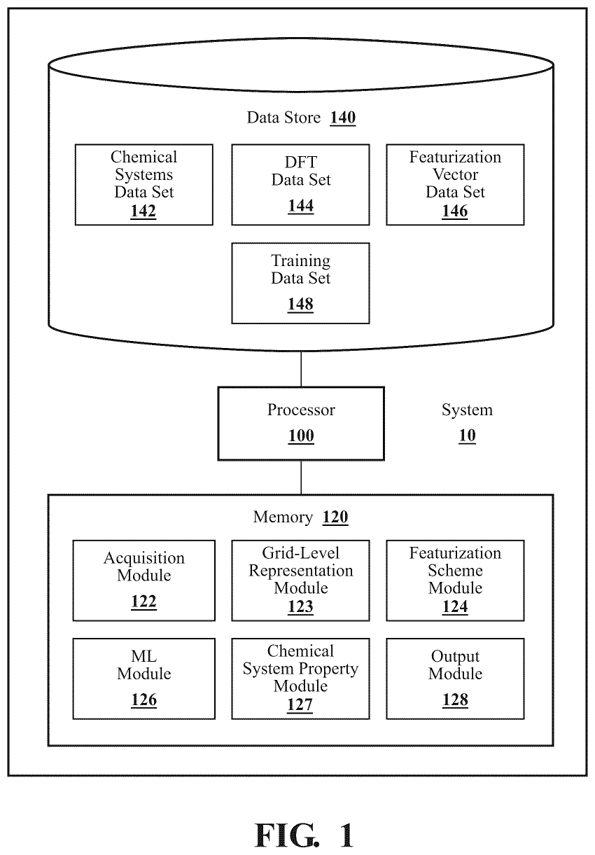

Referring to FIG. 1, a block diagram of a system 10 for determining the electronic structure of a chemical system according to the teachings of the present disclosure is shown. The system 10 is shown including a processor(s) 100, a memory 120, and a data store 140 communicably coupled to the processor(s) 100. It should be understood that the processor(s) 100 can be part of the system 10, or in the alternative, the system 10 can access the processor(s) 100 through a data bus or another communication path.

The memory 120 is configured to store an acquisition module 122, a grid-level representation module 123, a featurization scheme module 124, a ML module 126, a chemical system property module 127, and an output module 128 (hereafter also referred to as “modules 122-128”). The memory 120 is a random-access memory (RAM), read-only memory (ROM), a hard-disk drive, a flash memory, or other suitable memory for storing the modules 122-128. Also, the modules 122-128 are, for example, computer-readable instructions that when executed by the processor 100 cause the processor(s) to perform the various functions disclosed herein.

In some variations the data store 140 is a database, e.g., an electronic data structure stored in the memory 120 or another data store. Also, in at least one variation the data store 140 in the form of a database is configured with routines that can be executed by the processor 100 for analyzing stored data, providing stored data, organizing stored data, and the like. Accordingly, in some variations the data store 140 stores data used by the acquisition module 122 and/or the ML module 126. For example, and as shown in FIG. 1, in at least one variation the data store 140 stores a chemical system data set 142, a density functional theory (DFT) data set 144, and an optional featurization vector data set 146 (also referred to herein collectively as “data sets 142-146”). In addition, the data store 140 can store a training data set 148.

As used herein, the phrase “density functional theory” and the acronym “DFT” refer to a computational quantum mechanical modelling method used in physics, chemistry, and materials science to investigate the electronic structure (principally the ground state) of many-body systems such as atoms, molecules, and the condensed phases.

In some variations, the chemical system data set 142 includes a listing of chemical systems and the DFT electronic structure data set 144 includes DFT electronic structure data properly tagged to the chemical systems in the chemical system data set 142. Non-limiting examples of chemical systems in the chemical system data set 142 include crystalline materials (e.g., oxides, nitrides, sulfides, intermetallics, among others), amorphous materials, liquids, and gases. Non-limiting examples of the DFT electronic structure data in the DFT electronic structure data set 144 include grid-based electron density data for the chemical systems in the chemical system data set 142, atom-based electron density data for the chemical systems in the chemical system data set 142, chemical system structure data such as optimized atomic positions, lattice parameters, and other structural parameters of the chemical systems in the chemical system data set 142, grid-based and/or atom-based electronic structure data such as band structures, electronic density of states, and other electronic properties of the chemical systems in the chemical system data set 142, grid-based and/or atom-based energy data such as electronic energy, and other energy-related parameters of the chemical systems (e.g., total energy) in the chemical system data set 142, grid-based and/or atom-based vibrational data such as vibrational frequencies and mode information for the chemical systems in the chemical system data set 142, and other property data such as elastic constants, cohesive energy, and heats of formation for the chemical systems in the chemical system data set 142.

In some variations, and when stored in the data store 140, the featurization vector data set 146 includes featurization vectors of the grid-level representations of and properly tagged to the materials in the chemical system data set 142. In some variations, the featurization vectors are Gaussian-multipole (GMP) featurization vectors as disclosed in the journal article titled “A Universal Framework for Featurization of Atomistic Systems” by Lei et al., J. Phys. Chem. Lett. 2022, 13, 34, 7911-7919, which is incorporated in the present disclosure in its entirety by reference. In other variations, the data store 140 does not store the featurization vector data set 146. For example, in some variations the featurization scheme module 124 can include computer-readable instructions, that when executed by the processor(s) 100, cause the processor(s) 100 to calculate GMP featurization vectors for one or more of the materials in the chemical systems data set 142.

The training data set 148 includes training data, sometimes referred to as “ground-truth data” in the form of chemical systems, DFT electronic structure data, and featurization vectors. In at least one variation, the training data set 148 includes a plurality of chemical systems, grid-based DFT electronic structure data for the plurality of chemical systems, and grid-based GMP featurization vectors for the plurality of chemical system.

The acquisition module 122 can include instructions that function to control the processor(s) 100 to select a training data set 148 stored in the data store 140 and provide the selected training data set 148 to the ML module 126. The acquisition module 122 can also include instructions that function to control the processor(s) 100 to select a chemical system from the chemical systems data set 142 to the ML module 126.

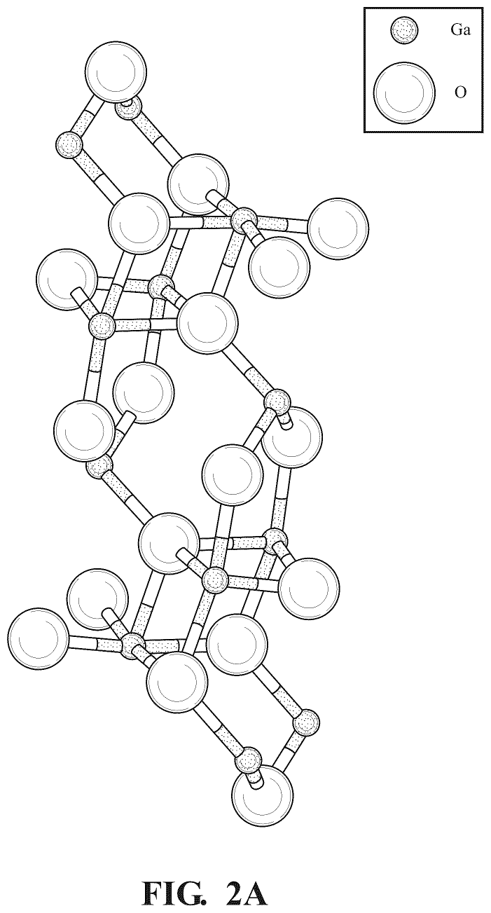

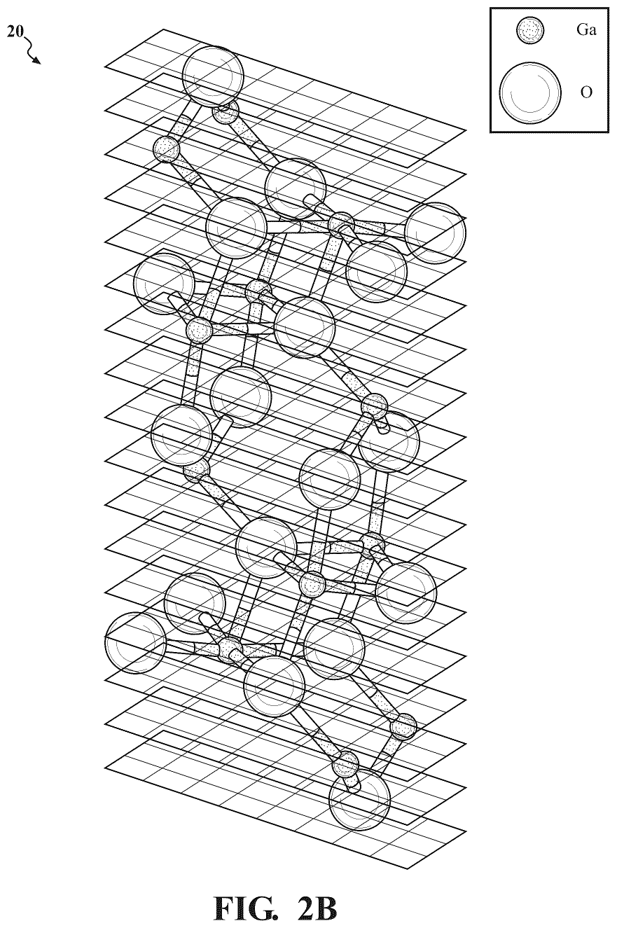

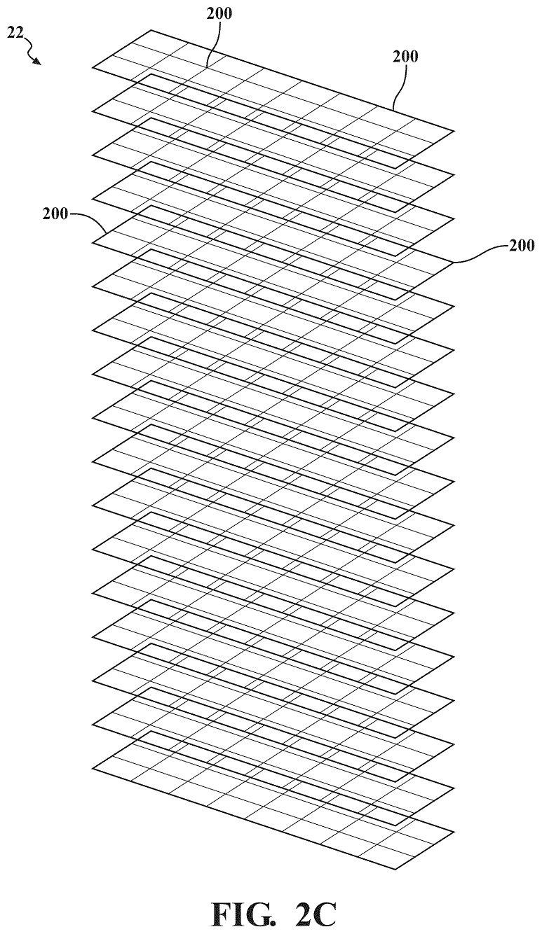

The grid-level representation module 123 can include instructions that function to control the processor 100 to generate or provide a grid-level representation of a selected chemical system. For example, and with reference to FIGS. 2A-2C, FIG. 2A shows an atom model for gallium oxide, i.e., a selected chemical system. FIG. 2B illustrates a grid 20 generated and/or provided by the grid-level representation module 123 and overlayed onto the atom model in FIG. 2A. And FIG. 2C illustrates the grid 20 in FIG. 2B with the atom model removed such that the grid 20 in FIG. 2B is a grid-level representation 22 of the selected chemical system in FIG. 2A. In this manner, a grid-level representation is generated and used to represent the physical structure of an atom model of a selected chemical system.

In some variations, the grid representation 22 is a uniform 3D grid representation of the selected chemical system, while in other variations the grid representation 22 is a non-uniform 3D grid representation. As used herein, the phrase “uniform 3D grid representation” refers to a grid representation with a grid 20 having equally spaced distances between grid points 200 and the phrase “nonuniform 3D grid representation” refers to a grid representation with a grid 20 having non-equal spaced distances between at least of portion of the grid points 200.

The featurization scheme module 124 can include instructions that function to control the processor 100 to generate featurization vectors for each of the grid points 200 of the grid-level representation 22. In some variations, the featurization scheme module 124 can include instructions that function to control the processor 100 to generate GMP featurization vectors for each of the grid points 200 of the grid-level representation 22 as described in the Lei et al. reference noted above. It should be understood that “featurization vectors” are ordered lists of numerical properties that represent the features (e.g., properties, positions, etc.) of an object or phenomenon.

The ML module 126 can include instructions that function to control the processor 100 to train the selected ML model using ground-truth data (e.g., using a selected training data set 148) such that the trained ML model can predict an electronic structure and/or one or more electronic structure properties of a selected chemical system. In some variations, the ML module 126 can include instructions that function to control the processor 100 to train the selected ML model using ground-truth data such that the ML model can predict DFT electronic structure data for a selected chemical system with grid-level GMP featurization vectors. Stated differently, the trained ML model is configured to predict one or more electronic structure properties of a chemical system selected from the chemical systems data set 142. The ML module 126 can also include instructions that function to control the processor 100 to predict DFT electronic structure data as described above for a chemical system selected from the chemical systems data set 142 and/or a chemical system provided to the system 10 via a user interface (not shown).

Non-limiting examples of the ML model include supervised regression ML models such as a linear regression model, a multiple linear regression model, a polynomial regression model, a decision tree regression model, a random forest regression model, a K-nearest neighbor regression model, and a neural network regression model among others.

The chemical system property module 127 can include instructions that function to control the processor 100 to predict one or more material properties of a selected chemical system as a function of the predicted one or more electronic structure properties of the selected from the chemical systems data set 142. In some variations, the chemical system property module 127 predicts a material property such as dielectric constant, bandgap energy, charge density distribution, dipole moment, Density of States (DOS) among others, using or as a function the electronic structure information predicted for each grid point of the grid-level representation of the selected chemical system.

The output module 128 can include instructions that function to control the processor(s) 100 to provide output (e.g., predictions) from the ML module 126 and/or the chemical system property module 127 to a user interface (known shown) and/or to the training data set 148.

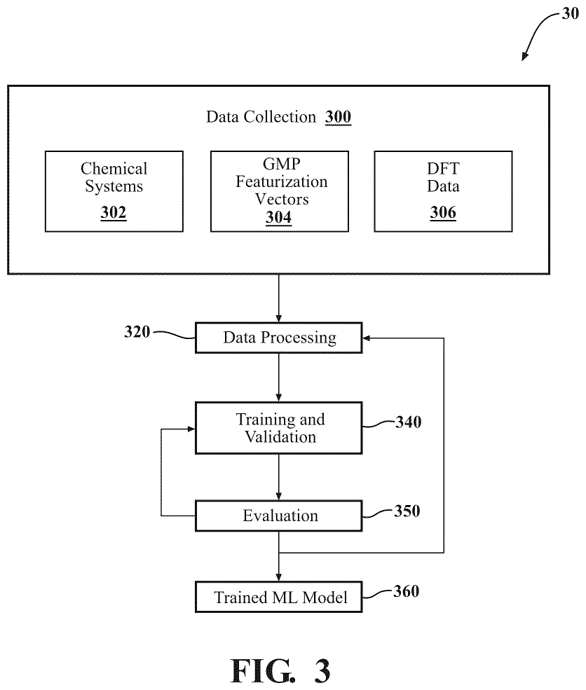

Referring now to FIG. 3, a flow chart for a method 30 for training a ML model to predict one or more electronic structure properties of a chemical system is shown. The method 30 provides a trained ML model at 360 via data collection 300, data processing 320, training and validation 340, and evaluation 350. It should be understood that the method 30 can be implemented by the system 10 according to the teachings of the present disclosure.

Data collection at 300 includes gathering various types of data necessary for training the machine learning model. The types of data can include chemical systems 302 and properly tagged GMP featurization vectors 304 and DFT electronic structure data 306 for the chemical systems 302. In some variations the GMP featurization vectors 304 are stored in the featurization vector data set 146. In the alternative, or in addition to, the GMP featurization vectors 304 are calculated for the chemical systems 302 (using a predefined featurization scheme) and provided for training the ML model without being stored in the featurization vector data set 146. Stated differently, and given that the GMP featurization vectors 304 can be computed/calculated efficiently, the GMP featurization vectors 304 are calculated “on the fly” and not stored in the data store 140.

Data processing at 320 prepares the collected data for use in training the ML model. Non-limiting examples of data processing steps or types include cleaning, normalizing, and/or transforming the collected data to ensure the data is in a suitable format for input to and analysis by the ML model.

Training and validation of the ML model are carried out using the processed data at 340 such that parameters of the ML model are adjusted to enhance performance and ensure the ML model more accurately predicts desired outcomes, e.g., more accurately predicts the electronic structure and/or one or more electronic structure properties for a selected chemical system.

Evaluation of the trained and validated ML model is conducted at 350 to determine the effectiveness of the ML model in predicting the electronic structure and/or one or more electronic structure properties for a selected chemical system. It should be understood that evaluation involves testing the ML model with new data and assessing the performance of the ML model against predefined criteria. In addition, results of the evaluation can be used to refine the ML model further if needed.

A trained ML model ready for deployment (use) is provided at 360. Stated differently, the trained ML model can now be used to predict the electronic structure and/or one or more electronic structure properties for a selected chemical system. Non-limiting examples of electronic structure properties predicted by the trained ML model include grid-based electron density data, lattice parameters, grid-based band structures, grid-based electronic density of states, grid-based electronic energy, grid-based vibrational frequencies, grid-based mode information, grid-based elastic constants, and/or grid-based cohesive energy.

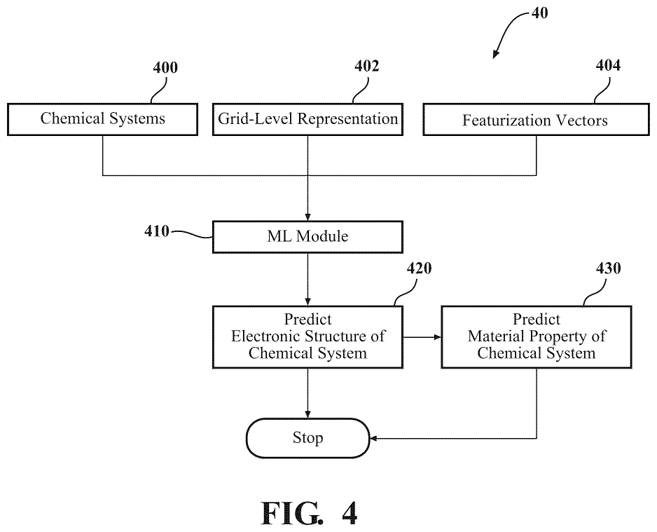

Referring to FIG. 4, a flow chart for a method 40 of predicting the electronic structure and/or one or more electronic structure properties of a chemical system is shown and described with reference to FIG. 1. The method 40 includes providing a selected chemical system at 400, a grid-level representation of the chemical system at 402, and GMP featurization vectors of the chemical system at 406 to the ML module 126 at 410. The ML module 126 includes a trained ML model as described above, and the trained ML model predicts the electronic structure and/or one or more electronic structure properties of the chemical system at 420. In some variations, the method 40 stops after predicting the electronic structure and/or one or more electronic structure properties of the chemical system at 420, while in other variations the method 40 proceeds to 430 and the system 10 predicts a material property for the chemical system before stopping.

In one example, the system 10 and methods 30, 40 are used to predict charge densities and LDOS for perovskite materials. Particularly, a uniform grid with one million grid points is generated for unit cells of perovskite material atomic structures. Also, GMP feature vectors are computed using the GMP featurization scheme discussed above, and DFT calculations are performed for a subset of the perovskite materials. The subset of perovskite materials and their corresponding grid-level charge density and LDOS are provided and used to train the ML model. Then the feature vectors of all the perovskite materials are used as input for the trained ML model, which predicts charge densities and LDOS for all of the perovskite materials. In some variations, other relevant physical properties such as energies, forces, phonon spectra, density of states, bandgaps, dielectric constants, and more are calculated using Kohn-Sham density functional theory algorithms/models and/or solid-state mechanics algorithms/models. It should be understood that the system 10 and methods 30, 40 overcome the problem, in this example, of thousands if not millions of CPU hours needed to calculate the charge densities and LDOS for all known perovskite materials using DFT calculations. That is, the systems and methods according to the teachings of the present disclosure predict the charge densities and LDOS for all known perovskite materials using or with orders or magnitude less CPU hours compared to DFT calculations.

While described as methods and illustrated with flowcharts, it should be understood that the present disclosure provides for systems that execute the methods described herein. For example, a system disclosed herein can include a processor, a memory communicably coupled to the processor and storing machine-readable instructions that, when executed by the processor, cause the processor to perform the methods and/or method steps disclosed herein, among others.

The preceding description is merely illustrative in nature and is in no way intended to limit the disclosure, its application, or uses. Work of the presently named inventors, to the extent it may be described in the background section, as well as aspects of the description that may not otherwise qualify as prior art at the time of filing, are neither expressly nor impliedly admitted as prior art against the present technology.

As used herein, the phrase at least one of A, B, and C should be construed to mean a logical (A or B or C), using a non-exclusive logical “or.” It should be understood that the various steps within a method may be executed in different order without altering the principles of the present disclosure. Disclosure of ranges includes disclosure of all ranges and subdivided ranges within the entire range.

The headings (such as “Background” and “Summary”) and sub-headings used herein are intended only for general organization of topics within the present disclosure and are not intended to limit the disclosure of the technology or any aspect thereof. The recitation of multiple variations or forms having stated features is not intended to exclude other variations or forms having additional features, or other variations or forms incorporating different combinations of the stated features.

The flowcharts and block diagrams in the figures illustrate the architecture, functionality, and operation of possible implementations of systems, methods, and computer program products according to various embodiments. In this regard, a block in the flowcharts or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved.

The systems, components and/or processes described above can be realized in hardware or a combination of hardware and software and can be realized in a centralized fashion in one processing system or in a distributed fashion where different elements are spread across several interconnected processing systems. Any kind of processing system or another apparatus adapted for carrying out the methods described herein is suited. A typical combination of hardware and software can be a processing system with computer-usable program code that, when being loaded and executed, controls the processing system such that it carries out the methods described herein. The systems, components and/or processes also can be embedded in a computer-readable storage, such as a computer program product or other data programs storage device, readable by a machine, tangibly embodying a program of instructions executable by the machine to perform methods and processes described herein. These elements also can be embedded in an application product which comprises the features enabling the implementation of the methods described herein and, which when loaded in a processing system, is able to carry out these methods.

Furthermore, arrangements described herein may take the form of a computer program product embodied in one or more computer-readable media having computer-readable program code embodied, e.g., stored, thereon. Any combination of one or more computer-readable media may be utilized. The computer-readable medium may be a computer-readable signal medium or a computer-readable storage medium. The phrase “computer-readable storage medium” means a non-transitory storage medium. A computer-readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer-readable storage medium would include the following: a portable computer diskette, a hard disk drive (HDD), a solid-state drive (SSD), a ROM, an EPROM or flash memory, a portable compact disc read-only memory (CD-ROM), a digital versatile disc (DVD), an optical storage device, a magnetic storage device, or any suitable combination of the foregoing. In the context of this document, a computer-readable storage medium may be any tangible medium that can contain or store a program for use by or in connection with an instruction execution system, apparatus, or device.

Generally, modules as used herein include routines, programs, objects, components, data structures, and so on that perform particular tasks or implement particular data types. In further aspects, a memory generally stores the noted modules. The memory associated with a module may be a buffer or cache embedded within a processor, a RAM, a ROM, a flash memory, or another suitable electronic storage medium. In still further aspects, a module as envisioned by the present disclosure is implemented as an ASIC, a hardware component of a system on a chip (SoC), as a programmable logic array (PLA), or as another suitable hardware component that is embedded with a defined configuration set (e.g., instructions) for performing the disclosed functions.

Program code embodied on a computer-readable medium may be transmitted using any appropriate medium, including but not limited to wireless, wireline, optical fiber, cable, radio frequency (RF), etc., or any suitable combination of the foregoing. Computer program code for carrying out operations for aspects of the present arrangements may be written in any combination of one or more programming languages, including an object-oriented programming language such as Java™, Smalltalk, C++, Python, or the like and conventional procedural programming languages, such as the “C” programming language or similar programming languages. The program code may execute entirely on the user's computer, partly on the user's computer, as a stand-alone software package, partly on the user's computer and partly on a remote computer, or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

As used herein, the terms “comprise” and “include” and their variants are intended to be non-limiting, such that recitation of items in succession or a list is not to the exclusion of other like items that may also be useful in the devices and methods of this technology. Similarly, the terms “can” and “may” and their variants are intended to be non-limiting, such that recitation that a form or variation can or may comprise certain elements or features does not exclude other forms or variations of the present technology that do not contain those elements or features.

The broad teachings of the present disclosure can be implemented in a variety of forms. Therefore, while this disclosure includes particular examples, the true scope of the disclosure should not be so limited since other modifications will become apparent to the skilled practitioner upon a study of the specification and the following claims. Reference herein to one variation, or various variations means that a particular feature, structure, or characteristic described in connection with a form or variation, or particular system is included in at least one variation or form. The appearances of the phrase “in one variation” (or variations thereof) are not necessarily referring to the same variation or form. It should also be understood that the various method steps discussed herein do not have to be carried out in the same order as depicted, and not each method step is required in each variation or form.

The foregoing description of the forms and variations has been provided for purposes of illustration and description. It is not intended to be exhaustive or to limit the disclosure. Individual elements or features of a particular form or variation are generally not limited to that particular form or variation, but, where applicable, are interchangeable and can be used in a selected form or variation, even if not specifically shown or described. The same may also be varied in many ways. Such variations should not be regarded as a departure from the disclosure, and all such modifications are intended to be included within the scope of the disclosure.

What is claimed is:

1. A system for predicting electronic structures for chemical systems, the system comprising:
a processor: and
a memory communicably coupled to the processor and storing machine-readable instructions that, when executed by the processor, cause the processor to:
train a machine learning (ML) regression module with chemical systems and corresponding electronic structures of the chemical systems;
generate a grid-level representation of a selected chemical system;
generate feature vectors for the grid-level representation; and
predict, using the trained ML regression model, an electronic structure for the selected chemical system.

2. The system according to claim 1, wherein the ML regression model is trained with density functional theory (DFT) data on the chemical systems and featurization vectors for grid points of grid-level representations of the chemical systems.

3. The system according to claim 2, wherein the DFT electronic structure data is selected from the group consisting of DFT charge density data and DFT local density of states (LDOS) data.

4. The system according to claim 3, wherein the DFT electronic structure data is the DFT charge density data.

5. The system according to claim 3, wherein the DFT electronic structure data is the DFT LDOS data.

6. The system according to claim 2, wherein the featurization vectors are GMP featurization vectors.

7. The system according to claim 2, wherein the grid-level representations are 3D grid-level representations.

8. The system according to claim 1, wherein generated grid-level representation of the selected chemical system is a generated 3D grid-level representation.

9. The system according to claim 8, wherein the generated 3D grid-level representation is a uniform 3D grid-level representation.

10. The system according to claim 8, wherein the generated 3D grid-level representation is a non-uniform 3D grid-level representation.

11. The system according to claim 1, wherein the selected chemical system is an oxide.

12. The system according to claim 1, wherein predicted electronic structure for the selected chemical system comprises predicted charge density and local density of states (LDOS) for grid points of the grid-level representation of the selected chemical system.

13. The system according to claim 12 further comprising calculating a chemical system property for the selected chemical system as a function of the predicted charge density and LDOS for grid points of the grid-level representation of the selected chemical system.

14. The system according to claim 13, wherein the predicted chemical system property is selected from the group consisting of a dielectric constant and a bandgap energy for the selected chemical system.

15. The system according to claim 14, wherein the predicted chemical system property is the dielectric constant.

16. The system according to claim 14, wherein the predicted chemical system property is the bandgap energy.

17. A system for predicting electronic structures for chemical systems, the system comprising:
a processor: and
a memory communicably coupled to the processor and storing machine-readable instructions that, when executed by the processor, cause the processor to:
train a machine learning (ML) regression module with grid-level Gaussian multipole (GMP) featurization vectors for chemical systems and corresponding grid-level density functional theory (DFT) data for the chemical systems;
generate a 3D grid-level representation of a selected chemical system;
generate a GMP featurization vectors for the grid-level representation; and
predict, using the ML regression model, grid-level DFT electronic structure data for the selected chemical system.

18. The system according to claim 17, wherein the DFT electronic structure data is at least one of DFT charge density data and DFT local density of states (LDOS) data.

19. The system according to claim 17, wherein predicted grid-level DFT electronic structure data for the selected chemical system comprises predicted charge density and local density of states (LDOS) for grid points of the grid-level representation of the selected chemical system.

20. A system for predicting electronic structures for chemical systems, the system comprising:
a processor: and
a memory communicably coupled to the processor and storing machine-readable instructions that, when executed by the processor, cause the processor to:
generate a 3D grid-level representation of a selected chemical system;
generate a Gaussian multipole (GMP) featurization vectors for the grid-level representation; and
predict, using a machine learning (ML) regression model trained with grid-level GMP featurization vectors and corresponding grid-level density functional theory (DFT) data for chemical systems, grid-level electronic structure data for the selected chemical system, wherein the corresponding DFT electronic structure data for the chemical systems comprises at least one of DFT charge density data and DFT local density of states (LDOS) data, and the predicted grid-level electronic structure data for the selected chemical system comprises at least of charge density data and LDOS data for the selected chemical system.
